(19) United States
(12) Patent Application Publication
(10) Pub. No.: US 2004/0242594 A1
(43) Pub. Date: Dec. 2, 2004

(54) Prodrugs of antidepressants and their use for treating depressions

(76) Inventors: Dan Peters, Malmo (SE); Carl-Emil Sandberg, Smorum (DK)

Correspondence Address: BIRCH STEWART KOLASCH & BIRCH, PO BOX 747, FALLS CHURCH, VA, 22040-0747, US

(21) Appl. No.: 10/489,657

(22) PCT Filed: Nov. 15, 2002
(86) PCT No.: PCT/DK02/00767

(30) Foreign Application Priority Data
Nov. 17, 2001 (DK) PA2001-01713

(51) Int. Cl. A61K 31/519; A61K 31/24; A61K 31/205

(52) U.S. Cl. 514/258.1; 514/554; 514/540

(57) ABSTRACT

This invention relates to prodrugs of antidepressants and the use of these prodrugs in a method for therapy and to pharmaceutical compositions comprising the prodrugs of the invention.

### TECHNICAL FIELD

This invention relates to prodrugs of antidepressants.

In other aspects the invention relates to the use of these prodrugs in a method for therapy and to pharmaceutical compositions comprising the prodrugs of the invention.

### BACKGROUND ART

Depression is a serious illness that affects more than ten million people in the USA alone. One of the greatest problems in treating depression is non-compliance with the treatment program. The reasons for non-compliance with taking prescription medicines vary among individuals and include: Feeling too depressed or embarrassed to take the prescription, becoming discouraged because the medicine may take as long as six weeks to take effect, misinformation about how anti-depressant medicine works or unwarranted fear of becoming addicted to it.

Furthermore, non-compliance—such as early discontinuation of the medicine because it is working—is likely to lead to relapse or recurrence of the depression.

Thus there is a continued strong need to find medications that give a better compliance for patients with depression.

### SUMMARY OF THE INVENTION

Therefore, in its first aspect, the invention provides a prodrug of an antidepressant having a hydroxy group or a carboxy group, which prodrug is a covalent conjugate of the antidepressant and either a carboxylic acid or an alcohol, or a pharmaceutically acceptable salt of said prodrug.

In its second aspect the invention provides a pharmaceutical composition comprising a therapeutically effective amount of a prodrug of the invention together with at least one pharmaceutically-acceptable carrier, excipient or diluent.

In a further aspect the invention provides a method of treatment, prevention or alleviation of a disease or a disorder or a condition of a living animal body, including a human, which disorder, disease or condition is which disease, disorder or condition is related to depression, which method comprises the step of administering to such a living animal body in need thereof a therapeutically effective amount of a compound of the invention.

Other objects of the invention will be apparent to the person skilled in the art from the following detailed description and examples.

The prodrug of the invention gives extended therapeutic effectiveness and permits less frequent dosing compared to the unconjugated antidepressant, thereby enhancing the possibility of compliance with the treatment for patients with depression.

### DETAILED DISCLOSURE OF THE INVENTION

In its first aspect, the invention provides a prodrug of an antidepressant having a hydroxy group or a carboxy group, which prodrug is a covalent conjugate of the antidepressant and either a carboxylic acid or an alcohol, or a pharmaceutically acceptable salt of said prodrug.

In a special embodiment, the prodrug and the carboxylic acid or the alcohol are conjugated via an ester bond, a carbamate bond, or a carbonate bond.

In its second aspect, the invention relates to a prodrug of an antidepressant having a hydroxy group or a carboxy group, which prodrug is an ester derivative formed between

either the hydroxy group of the antidepressant and a carboxylic acid or the carboxy group of the antidepressant and an alcohol, or a pharmaceutically acceptable salt of said prodrug.

In one embodiment, the antidepressant having a hydroxy group or a carboxy group is an antidepressant having a hydroxy group. In a second embodiment, the antidepressant having a hydroxy group or a carboxy group is an antidepressant having a carboxy group. In a further embodiment, the antidepressant having a hydroxy group may have one or more hydroxy groups. In a special embodiment, the antidepressant having a hydroxy group has one hydroxy group. In a further embodiment, the antidepressant having a carboxy group may have one or more carboxy groups. In a special embodiment, the antidepressant having a carboxy group has one carboxy group.

The antidepressant having a hydroxy group or a carboxy group may be selected from the group of tricyclic antidepressants, the group of monoamine oxidase inhibitors (MAOIs), the group of reversible MAO type A inhibitors (RIMAs), the group of tetracyclic antidepressants, the group of selective serotonin re-uptake inhibitors (SSRIs), the group of serotonin noradrenaline re-uptake inhibitors (SNRIs), the group of noradrenaline re-uptake inhibitors (NARIs), the group of noradrenergic specific serotonergic antidepressants (NaSSAs), or any other antidepressant.

In a further embodiment, the antidepressant having a hydroxy group is selected from the group of compounds consisting of: opipramol (4-[3-(5H-dibenz[b,f]azepin-5-yl)propyl]-1-piperazine-ethanol (dihydrochloride)), venlafaxine (venlafexine), befloxatone ((R)-5-(methoxymethyl)-3-(p[(R)-4,4,4-trifluoro-3-hydroxybutoxy]phenyl]-2-oxazolidinone), MDL-72394 ((E)-β-fluoromethylene-m-tyrosine; or (E)-2-amino-4-fluoro-3-(m-hydroxyphenyl)-3-butenoic acid), danitracen (10-(1-methyl-4-piperidylidene)-9,10-dihydro-9-anthrol), ciclazindol (10-(3-chlorophenyl)-2,3,4,10-tetrahydro-pyrimido[1,2-a]indol-10-ol), RS-8359+((±)-6,7-dihydro-7-hydroxy-5H-cyclopenta[d]pyrimidinyl-4-aminobenzonitrile), 2 hydroxyimipramine (5-[3-(dimethylamino)propyl]-10,11-dihydro-5H-dibenz[b,f]azepin-2-ol), 2-hydroxydesipramine (5-[3-(methylamino)propyl]-10,11-dihydro-5H-dibenz[b,f]azepin-2-ol), 10-hydroxyamitriptyline (5,11-dihydro-10-hydroxy-N,N-dimethyl-dibenzo[a,d]cycloheptene-Δ^{5,γ}-propylamine), 10-hydroxynortriptyline (5,11-dihydro-10-hydroxy-N-methyl-dibenzo[a,d]cycloheptene-Δ^{5,γ}-propylamine), trazium esilate (1-(p-chlorophenyl)-1,2-dihydro-1-hydroxy-as-triazino[6,1-a]isoquinolin-5-ium ethanesulfonate), oxaprotiline ((±)-α-[(methylamino)methyl]-9,10-ethanoanthracene-9-(10H)-ethanol), levoprotiline (the (R)-(−) enantiomer, (R)-(−)α-[(methylamino)methyl]-9,10-ethanoanthracene-9-(10H)-ethanol, or the (S)-(+) enantiomer, (S)-(+)α-[(methylamino)methyl]-9,10-ethanoanthracene-9-(10H)-ethanol), cilobamine (cis-2-(3,4-dichlorophenyl)-3-isopropylamino-bicyclo[2.2.2]octan-2-ol (methanesulfonate)), flesinoxan ((±)-(S)-4-fluoro-N-[2-[4-(2-2-hydromethyl)-1,4-benzodioxan-5-yl]-1-piperarinyl]ethyl] benzamide), ifoxetine ((±)-cis-4-(2,3-xylyloxy)-3-piperidinol (sulfate)), flerobuterol (α-[tert-butylamino]methyl-o-fluorobenzyl alcohol), BRL 14342 (1-amino-3-(m-chlorophenyl)-N,N-dimethyl-3-phenyl-2-propanol), bipenamol (o-[(α-amino-o-tolyl)thio]benzyl alcohol), cyprolidol (α,α-diphenyl-α-[2-(4-pyridyl) cyclopropyl]-methanol), and dazadrol (α-(p-chlorophenyl)-α-(2-imidazolinyl)-2-pyridine-methanol (Z)-2-butenedionate).

In a special embodiment, the antidepressant having a hydroxy group is selected from the group of compounds consisting of: opipramol, venlafaxine, and befloxatone.

In a further special embodiment, the antidepressive having a hydroxy group is venlafaxine.

Venlafaxine (1-[2-dimethylamino-1-(4-methoxy-phenyl)-ethyl]-cyclohexanol) and its preparation is i.a. described in U.S. Pat. No. 4,535,186. Venlafaxine is a racemic mixture of (−) isomer and the (+) isomer. In a special embodiment the antidepressive having a hydroxy group is the racemate of venlafaxine. In a further embodiment the antidepressive having a hydroxy group is the (−) isomer of venlafaxine. In a still further embodiment the antidepressive having a hydroxy group is the (+) isomer of venlafaxine.

In a further embodiment, the antidepressant having a hydroxy group is a compound of the general formula (Ia) or (Ib),

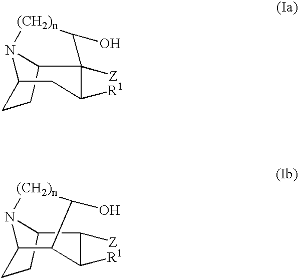

or a pharmaceutically acceptable addition salt thereof or the N-oxide thereof wherein Z is hydrogen or —COOR²; R² is alkyl, alkenyl, alkynyl, cycloalkylalkyl, or arylalkyl; R¹ is alkyl, alkenyl, alkynyl, aryl, or arylalkyl; where said aryl groups are optionally substituted one or more times with substituents selected from the group consisting of halogen, CF₃, CN, alkoxy, cycloalkoxy, alkyl, cycloalkyl, alkenyl, alkynyl, amino, alkylamino, dialkylamino and nitro; and n is 1, 2, 3, or 4.

Compounds of the general formula (Ia) or (Ib) and their preparation are described in WO 97/16451 (NeuroSearch A/S).

In a special embodiment, the antidepressant having a hydroxy group is a compound of the general formula (Ia) or (Ib) as above, wherein

Z is hydrogen; R¹ is aryl optionally substituted one or more times with substituents selected from the group consisting of halogen, CF₃, CN, alkoxy, cycloalkoxy, alkyl, cycloalkyl, alkenyl, alkynyl, amino, alkylamino, dialkylamino and nitro; and n is 1, 2, 3, or 4. In a special embodiment thereof, R¹ is chlorophenyl or-dichlorophenyl.

In a second special embodiment, the antidepressant having a hydroxy group is a compound of the general formula (Ia) or (Ib) as above, wherein R² is alkyl, alkenyl, or alkynyl.

In a further special embodiment, the antidepressant having a hydroxy group is a compound of the general formula (Ia) or (Ib) as above and the carboxylic acid forming the ester derivative is a C₇₋₃₀-carboxylic acid, such as a C₇₋₂₀-carboxylic acid.

In a still further special embodiment, the antidepressant having a hydroxy group is selected from the group of compounds consisting of:

(1S,3S,4S,5R/S,8R)-3-(3,4-dichlorophenyl)-7-azatricyclo[5.3.0.0]decan-5-ol;

(1S,3S,4S,5S,8R)-3-(3,4-dichlorophenyl)-7-azatricyclo[5.3.0.0]decan-5-ol;

(1S,3S,4S,5S,8R)-3-(3,4-dichlorophenyl)-7-azatricyclo[5.3.0.0]decan-5-ol;

(1R,3R.4R,5R/S,8S)-3-(3,4-dichlorophenyl)-7-aza-tricyclo[5.3.0.0^{4,8}]decan-5-ol;

(1R,3R.4R,5R,8S)-3-(3,4-dichlorophenyl)-7-aza-tricyclo[5.3.0.0^{4,8}]decan-5-ol; and

(1R,3R,4R,5S,8S)-3-(3,4-Dichlorophenyl)-7-azatricyclo-[5.3.0.0^{4,8}]-decan-5-ol.

In a further embodiment of the invention, the carboxylic acid forming the ester derivative is a C₁₋₃₀-carboxylic acid. In a special embodiment, the carboxylic acid forming the ester derivative is a C₂₋₂₀-carboxylic acid. In a further special embodiment, the carboxylic acid forming the ester derivative is a C₇₋₂₀-carboxylic acid, such as a C₁₀₋₂₀-carboxylic acid.

In a special embodiment, the carboxylic acid forming the ester derivative is selected from the group consisting of octanoic acid, decanoic acid, lauric acid, myristic acid, and palmitic acid. In a further special embodiment, the carboxylic acid forming the ester derivative is selected from the group consisting of hexanoic acid, heptanoic acid, octanoic acid, decanoic acid, lauric acid, tridecanoic acid, myristic acid, pentadecanoic acid, and palmitic acid.

In a further embodiment, the prodrug is selected from the list consisting of:

(1R,3R,4R,5R,8S)-3-(3,4-dichlorophenyl)-7-aza-tricyclo[5.3.0.0^{4,8}]decan-5-yl octanoate;

(1R,3R,4R,5R,S)-3-(3,4-dichlorophenyl)-7-aza-tricyclo[5.3.0.0^{4,8}]decan-5-yl decanoate;

(1R,3R,4R,5R,8S)-3-(3,4-dichlorophenyl)-7-aza-tricyclo[5.3.0.0^{4,8}]decan-5-yl laurate;

(1R,3R,4R,5R,8S)-3-(3,4-dichlorophenyl)-7-aza-tricyclo[5.3.0.04t)decan-5-yl myristate;

(1R,3R,4R,5R,8S)-3-(3,4-dichlorophenyl)-7-aza-tricyclo[5.3.0.0^{4,8}]decan-5-yl palmitate;

(1S,3S,4S,5S,8R)-3-(3,4-dichlorophenyl)-7-aza-tricyclo[5.3.0.0^{4,8}]decan-5-yl octanoate;

(1S,3S,4S,5S,8R)-3-(3,4-dichlorophenyl)-7-aza-tricyclo[5.3.0.0^{4,8}]decan-5-yl decanoate;

(1 S,3S,4S,5S,8R)-3-(3,4-dichlorophenyl)-7-aza-tricyclo[5.3.0.0^{4,8}]decan-5-yl laurate;

(1S,3S,4S,5S,8R)-3-(3,4-dichlorophenyl)-7-aza-tricyclo[5.3.0.0^{4,8}]decan-5-yl myristate; and

(1S,3S,4S,5S,8R)-3-(3,4-dichlorophenyl)-7-aza-tricyclo[5.3.0.0^{4,8}]decan-5-yl palmitate.

In a further embodiment, the prodrug is selected from the list consisting of:

1-[2-Dimethylamino-1-(4-methoxy-phenyl)-ethyl]-cyclohexanyl tridecanoate,

1-[2-Dimethylamino-1-(4-methoxy-phenyl)-ehyl]-cyclohexanyl pentadecanoate,

1-[2-Dimethylamino-1-(4-methoxy-phenyl)-ethyl]-cyclohexanyl heptadecanoate,

1-[2-Dimethylamino-1-(4-methoxy-phenyl)-ethyl]-cyclohexanyl eicosanoate, and

1-[2-Dimethylamino-1-(4-methoxy-phenyl)-ethyl]-cyclohexanyl heptanoate

In a further embodiment, the antidepressant having a carboxy group is selected from the group of compounds consisting of: MDL-72394 ((E)-β-fluoromethylene-m-tyrosine; or (E)-2-amino-4-fluoro-3-(m-hydroxyphenyl)-3-butenoic acid), amineptine (N-(10,11-dihydro-5H-dibenzo[a,d]cyclohepten-5-yl)-7-amino-heptanoic acid, tianeptine (N-(3-chloro-6,11-dihydro-6-methyl-bibenzo[c,f][1,2]thia-zepin-11-yl)-7-amino-heptanoic acid S,S-dioxide), and zafuleptine ((±)-7-[(p-fluorobenzyl)amino]-8-methylnonanoic acid).

In a special embodiment, the alcohol forming the ester derivative is a C₂₋₂₀-alcohol. In a more special embodiment, the alcohol forming the ester derivative is selected from the group consisting of: octyl alcohol, decyl alcohol, dodecyl alcohol, tetradecyl alcohol, and hexadecyl alcohol.

In a special embodiment of the invention, the activity of the prodrug is less than 10% of the activity of the antidepressant. In a further embodiment, the activity of the prodrug is less than 5% of the activity of the antidepressant. In a still further embodiment, the activity of the prodrug is less than 1% of the activity of the antidepressant.

Definitions

In the context of this invention halogen represents a fluorine, a chlorine, a bromine or an iodine atom.

Alkyl means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred-groups.

Cycloalkyl means cyclic alkyl of three to seven carbon atoms, including but not limited to cyclopropyl, cyclobutyl, cyclopentyl, and cyclohexyl.

Alkenyl means a group of from two to six carbon atoms, including at least one double bond, for example, but not limited to ethenyl, 1- or 2-propenyl, or 1-, 2-, or 3-butenyl.

Alkynyl means a group of from two to six carbon atoms, including at least one triple bond, for example, but not limited to ethynyl, 1-, 2-propynyl, or 1-, 2- or 3-butynyl.

Cycloalkoxy means O-cycloalkyl, wherein cycloalkyl is as defined above.

Cycloalkylalkyl means cycloalkyl as above and alkyl as above, meaning for example, cyclopropylmethyl.

Aryl is a carbocyclic aromatic ring system such as phenyl or naphthyl (1-naphthyl or 2-naphthyl).

Carboxylic acid means a straight or branched, saturated or unsaturated acid with from one to thirty carbon atoms, including but not limited to formic, acetic, propionic, butyric, isobutyric, valeric, heptanoic, octanoic, nonanoic, decanoic, and undecanoic acid. Included are also fatty acids having from 12 to 26 carbon atoms, such as unbranched naturally occurring fatty acids, including but not limited to C12:0 (lauric acid), C14:0 (myristic acid), C16:0 (palmitic acid), C16:1 (palmitoleic acid), C16:2, C18:0 (stearic acid), C18:1 (oleic acid), C18:3-6, C18:4-3, C20:1, C20:2-6, C20:3-6, C20:4-3; C20:4-6, C20:5-3, C22:1, C22:4-6, C22:5-6, C22:5-3, C22:6-3 and C24:1-9. Included are also fatty acids having from 13 to 19 carbon atoms, such as unbranched tridecanoic acid, pentadecanoic acid, heptadecanoic acid and nonadecanoic acid.

Alcohol means a straight or branched, saturated or unsaturated alcohol with from one to thirty carbon atoms, including but not limited to an alcohol having the same alkyl group as the above mentioned carboxylic acid.

The prodrugs of this invention may exist in unsolvated as well as in solvated forms with pharmaceutically acceptable solvents such as waters ethanol and the like. In general, the solvated forms are considered equivalent to the unsolvated forms for the purposes of this invention.

It will be appreciated by those skilled in the art that some prodrug of the present invention contain chiral centres and that such compounds exist in the form of isomers. The invention includes all such isomers and any mixtures thereof including racemic mixtures.

The prodrugs of the invention and their pharmaceutically acceptable derivatives may be prepared by any method known in the art for the preparation of compounds of analogous structure, and as shown in the representative examples which follow.

Pharmaceutically Acceptable Salts

The chemical prodrug of the invention may be provided in any form suitable for the intended administration. Suitable forms include pharmaceutically (i.e. physiologically) acceptable salts of the prodrug of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Labelled Prodrugs

The prodrugs of the invention may be used in their labelled or unlabelled form. In the context of this invention “label” stands for the binding of a marker to the compound of interest that will allow easy quantitative detection of said compound.

The labelled prodrugs of the invention may be useful as diagnostic tools, radio tracers, or monitoring agents in various diagnostic methods, and for in vivo receptor imaging.

The labelled prodrug of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled prodrug of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

Methods of Preparation

The prodrugs of the invention may be prepared by conventional methods for chemical synthesis, e.g. those described in the working examples. The starting materials for the processes described in the present application are known or may readily be prepared by conventional methods from commercially available chemicals.

For example, the ester derivative formed between the hydroxy group of the antidepressant and a carboxylic acid may be formed by reaction the antidepressant with the acid chloride, or the acid anhydride, or its carbbxyimidazolyl derivative, or the carboxylic acid catalysed by various acids. The invention includes all such end products, irrespective of how the ester derivative is formed.

The end product of the reaction described herein may be isolated by conventional techniques, e.g. by extraction, crystallisation, distillation, chromatography, etc.

Test Methods

The activity of prodrug and the released antidepressant as well as the release rate of the antidepressant from the prodrug can be measured by conventional methods in the art.

Pharmaceutical Compositions

In another aspect the invention provides novel pharmaceutical compositions comprising a therapeutically effective amount of a prodrug of the invention.

While a prodrug of the invention for use in therapy may be administered in the form of the raw chemical compound, it is preferred to introduce the active ingredient (optionally in the form of a physiologically acceptable salt) in a pharmaceutical composition together with one or more adjuvants, excipients, carriers, buffers, diluents, and/or other customary pharmaceutical auxiliaries.

In a preferred embodiment, the invention provides pharmaceutical compositions comprising a prodrug of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof. In a further embodiment, the invention provides pharmaceutical compositions comprising more than one prodrug of the invention, such as two different prodrugs of the invention.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The prodrug of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The prodrug of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a prodrug of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The prodrug according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. Administration by injection may e.g. be subcutaneous, intramuscular or intradermal. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the prodrug of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the prodrug used, the antidepressant being part of the prodrug, and on the nature and severity of the disease being treated, and is within the discretion of the physician, and may be varied by titration of the dosage to the particular circumstances of this invention to pro duce the desired therapeutic effect.

Preferably, the active ingredient may be administered in doses once or twice a week, once or twice every two weeks, once or twice every three weeks, or once or twice every month.

Methods of Therapy

In another aspect the invention provides a method for the treatment, prevention or alleviation of a disease or a disorder or a condition of a living animal body, including a human, which disease, disorder or condition is related to depression, and which method comprises administering to such a living animal body, including a human, in need thereof an effective amount of the prodrug of the invention. In a special embodiment, the effective amount of prodrug may be sum of more than one prodrug of the invention, such as two different prodrugs of the invention.

In a more preferred embodiment the invention provides a method of treating, prevention or alleviating depression, depressive disorders, major depression disorder, dysthymic disorder (dysthymia), bipolar affective disorder, mood disorder, or postnatal depression.

Furthermore, due to the neurotransmitter reuptake inhibitory activity of many of the antidepressants having a hydroxy or a carboxy group, many of the prodrugs of the invention are useful for treating, prevention or alleviating obsessive compulsive disorders, panic disorders, memory deficits, attention deficit hyperactivity disorder, obesity, anxiety, eating disorders, alcoholism, pain, pseudodementia, Ganser's syndrome, migraine pain, bulimia, pre-menstrual syndrome, late luteal phase syndrome, tobacco abuse, post-traumatic syndrome, memory loss, dementia of ageing, social phobia, chronic fatigue syndrome, anorexia nervosa, disorders of sleep, autism Parkinson's disease, Parkinsonism, narcolepsy, drug addition or misuse, senile dementia, presenile dementia, and Alzheimer's disease.

It is at present contemplated that suitable dosage ranges are equivalent to 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge. A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

Synthesis of Prodrugs

General

All reactions involving air sensitive reagents or intermediates were performed under nitrogen and in anhydrous solvents. Magnesium sulphate was used as drying agent in the workup-procedures and solvents were evaporated under reduced pressure.

Method A

1-[2-Dimethylamino-1-(4-methoxy-phenyl)-ethyl]-cyclohexanyl tridecanoate hydrochloric acid salt

A mixture of 1-[2-Dimethylamino-1-(4-methoxy-phenyl)-ethyl]-cyclohexanol hydrochloride (venlafaxine hydrochloric acid salt) (2.5 g, 8.0 mmol), tridecanoic acid chloride (2.98 g, 12.0 mmol), triethylamine (2.8 ml, 20 mmol) and THF (50 ml) was stirred for 0.5 h at room temperature. Water was added (200 ml) and the mixture was extracted with diethylether (2×50 ml). The organic phase was washed with water (2×50 ml). The final product was isolated as an oil in quantitative yield (3.8 g, 100%). The free base was converted to the corresponding hydrochloric acid salt, mp 136.50C.

1-[2-Dimethylamino-1-(4-methoxy-phenyl)ethyl]-cyclohexanyl pentadecanoate hydrochloric acid salt

Was prepared according to method A, from pentadecanoic acid chloride. Mp 137.3° C.

1-[2-Dimethylamino-1-(4-methoxy-phenyl)-ethyl]-cyclohexanyl heptadecanoate hydrochloric acid salt

Was prepared according to method A, from heptadecanoic acid chloride. Mp 137.6° C.

1-[2-Dimethylamino-1-(4-methoxy-phenyl)-ethyl]-cyclohexanyl eicosanoate hydrochloric acid salt

Was prepared according to method A, from eicosanoic acid chloride. Mp 138.1-147.7° C.

1-[2-Dimethylamino-1-(4-methoxy-phenyl)-ethyl]-cyclohexanyl heptanoate hydrochloric acid salt

Was prepared according to method A, from heptanoic acid chloride. Mp 138.1-147.7° C.

### Example 2

Synthesis of further prodrugs.

The following prodrugs are all prepared according to method A:

1-[2-Dimethylamino-1-(4-methoxy-phenyl)-ethyl]-cyclohexanyl acetate hydrochloric acid salt from acetyl chloride.

1-[2-Dimethylamino-1-(4-methoxy-phenyl)-ethyl]-cyclohexanyl propionate hydrochloric acid salt from propionyl chloride.

1-[2-Dimethylamino-1-(4-methoxy-phenyl)-ethyl]-cyclohexanyl butyrate hydrochloric acid salt from butyryl chloride.

1-[2-Dimethylamino-1-(4-methoxy-phenyl)-ethyl]-cyclohexanyl isobutyrate hydrochloric acid salt from isobutyryl chloride.

1-[2-Dimethylamino-1-(4-methoxy-phenyl)-ethyl]-cyclohexanyl pentanoate hydrochloric acid salt from pentanoic acid chloride.

1-[2-Dimethylamino-1-(4-methoxy-phenyl)-ethyl]-cyclohexanyl hexanoate hydrochloric acid salt from hexanoic acid chloride.

1-[2-Dimethylamino-1-(4-methoxy-phenyl)-ethyl]-cyclohexanyl octanoate hydrochloric acid salt from octanoic acid chloride.

1-[2-Dimethylamino-1-(4-methoxy-phenyl]ethyl]-cyclohexanyl nonanoate hydrochloric acid salt from nonanoic acid chloride.

1-[2-Dimethylamino-1-(4-methoxy-phenyl)-ethyl]-cyclohexanyl decanoate hydrochloric acid salt from decanoic acid chloride.

1-[2-Dimethylamino-1-(4-methoxy-phenyl)-ethyl]-cyclohexanyl undecanoate hydrochloric acid salt from undecanoic acid chloride.

1-[2-Dimethylamino-1-(4-methoxy-phenyl)-ethyl]cyclohexanyl dodecanoate hydrochloric acid salt from dodecanoic acid chloride.

1-[2-Dimethylamino-1-(4-methoxy-phenyl)-ethyl]-cyclohexanyl tetradecanoate hydrochloric acid salt from tetradecanoic acid chloride.

1-[2-Dimethylamino-1-(4-methoxy-phenyl)-ethyl]-cyclohexanyl hexadecanoate hydrochloric acid salt from hexadecanoic acid chloride.

1-[2-Dimethylamino-1-(4-methoxy-phenyl)-ethyl]-cyclohexanyl octadecanoate hydrochloric acid salt from octadecanoic acid chloride.

1-[2-Dimethylamino-1-(4-methoxy-phenyl)-ethyl]-cyclohexanyl nonadecanoate hydrochloric-acid salt from nonadecanoic acid chloride.

1. A prodrug of an antidepressant having a hydroxy group or a carboxy group, which prodrug is a covalent conjugate of the antidepressant and either a carboxylic acid or an alcohol, or a pharmaceutically acceptable salt of said prodrug.

2. A prodrug of an antidepressant having a hydroxy group or a carboxy group, which prodrug is an ester derivative formed between
either the hydroxy group of the antidepressant and a carboxylic acid or the carboxy group of the antidepressant and an alcohol, or a pharmaceutically acceptable salt of said prodrug.

3. A prodrug according to claim 2, wherein the antidepressant having a hydroxy group is selected from the group of compounds consisting of:
opipramol, venlafaxine, befloxatone, (E)-β-fluoromethylene-m-tyrosine, danitracen, ciclazindol, (±)-6,7-dihydro-7-hydroxy-5H-cyclopenta[d]pyrimidinyl-4-aminobenzonitrile, 2-hydroxyimipramine, 2-hydroxydesipramine, 10-hydroxyamitriptyline, 10-hydroxynortriptyline, trazium esilate, oxaprotiline, levoprotiline, cilobamine, flesinoxan, ifoxetine, flerobuterol, 1-amino-3-(m-chlorophenyl)-N,N-dimethyl-3-phenyl-2-propanol, bipenamol, cyprolidol, and dazadrol.

4. A prodrug according to claim 3, wherein the antidepressant having a hydroxy group is selected from the group of compounds consisting of:
opipramol, venlafaxine, and befloxatone.

5. A prodrug according to claim 2, wherein the antidepressant having a hydroxy group is venlafaxine.

6. A prodrug according to any one of claims 3-5, wherein the carboxylic acid forming the ester derivative is a C₂₋₂₀-carboxylic acid.

7. A prodrug according to claim 6, wherein the carboxylic acid forming the ester derivative is selected from the group consisting of:
hexanoic acid, heptanoic acid, octanoic acid, decanoic acid, lauric acid, tridecanoic acid, myristic acid, pentadecanoic acid, and palmitic acid.

8. A prodrug according to any one of claims 3-7, selected from the list consisting of:
1-[2-Dimethylamino-1-(4-methoxy-phenyl)-ethyl]-cyclohexanyl tridecanoate,
1-[2-Dimethylamino-1-(4-methoxy-phenyl)-ethyl]-cyclohexanyl pentadecanoate,
1-[2-Dimethylamino-1-(4-methoxy-phenyl)-ethyl]-cyclohexanyl heptadecanoate,
1-[2-Dimethylamino-1-(4-methoxy-phenyl)-ethyl]-cyclohexanyl eicosanoate, and
1-[2-Dimethylamino-1-(4-methoxy-phenyl)-ethyl]-cyclohexanyl heptanoate.

9. A prodrug according to claim 2, wherein the antidepressant having a carboxy group is selected from the group of compounds consisting of: (E)-β-fluoromethylene-m-tyrosine, amineptine, tianeptine, and zafuleptine.

10. A prodrug according to claim 9, wherein the alcohol forming the ester derivative is a C₂₋₂₀-alcohol.

11. A prodrug according to claims 9 or 10, wherein the alcohol forming the ester derivative is selected from the group consisting of: octyl alcohol, decyl alcohol, dodecyl alcohol, tetradecyl alcohol, and hexadecyl alcohol.

12. A pharmaceutical composition, comprising a therapeutically effective amount of a prodrug of any one of claims 1-11, together with at least one pharmaceutically acceptable carrier, excipient or diluent.

13. The use of a prodrug according to any one of claims 1-11, for the manufacture of a pharmaceutical composition for the treatment, prevention or alleviation of a disease, disorder or condition related to depression.

14. The use according to claim 13, wherein the disease, disorder or condition related to depression is selected from depression, depressive disorders, major depression disorder, dysthymic disorder (dysthymia), bipolar affective disorder, mood disorder, or postnatal depression.

15. A method for treatment, prevention or alleviation of a disease, disorder condition of a living animal body, including a human, which disease, disorder or condition is related to depression, which method comprises the step of administering to such a living animal body in need thereof a therapeutically effective amount of a prodrug according to any one of claims 1-11.

16. The method of claim 15, wherein the disease, disorder or condition related to depression is selected from depression, depressive disorders, major depression disorder, dysthymic disorder (dysthymia), bipolar affective disorder, mood disorder, or postnatal depression.
